(19) United States
(12) Patent Application Publication
(10) Pub. No.: US 2015/0045829 A1
(43) Pub. Date: Feb. 12, 2015

(54) DEVICE AND METHOD FOR APPETITE SUPPRESSION AND WEIGHT LOSS MANAGEMENT

(71) Applicant: David ASLEY, Smith Town, NY (US)

(72) Inventor: David ASLEY, Smith Town, NY (US)

(21) Appl. No.: 14/163,633

(22) Filed: Jan. 24, 2014

Related U.S. Application Data
(60) Provisional application No. 61/863,468, filed on Aug. 8, 2013.

(51) Int. Cl. A61F 5/00

(52) U.S. Cl. CPC A61F 5/0009
USPC 606/201

(57) ABSTRACT

A device is provided for mounting on a wearer to effect weight loss in the wearer. A central body is connected to a belt for mounting the central body on the abdomen of the wearer and a support structure is provided for stabilizing a position of the central body in relation to the body of the wearer.

### CROSS-REFERENCES TO RELATED APPLICATIONS

This application claims the benefit of prior filed U.S. provisional Application No. 61/863,468, filed Aug. 8, 2013, pursuant to 35 U.S.C. 119(e), the disclosure of which is incorporated herein by reference.

### BACKGROUND OF THE INVENTION

The invention relates to a device and a method for appetite suppression and weight loss maintenance.

The following discussion of related art is provided to assist the reader in understanding the advantages of the invention, and is not to be construed as an admission that this related art is prior art to this invention.

A device and method for appetite suppression is described in U.S. Pat. No. 4,592,342. The described device includes a central body having a protuberance and a belt for mounting the central body on a human being so that the protuberance is located about three fingers above the navel of the wearer, The protuberance is intended to displace the abdomen inwardly, thereby compressing the stomach to create a feeling of fullness.

A disadvantage of the device described in U.S. Pat. No. 4,592,342 is that in particular in obese persons, the device causes an uncomfortable wearing experience and does not have the expected appetite suppressing effect.

It would therefore be desirable and advantageous to provide an improved device for appetite suppression, which provides a more comfortable wearing experience and is more effective in suppressing appetite.

### SUMMARY OF THE INVENTION

The inventor of the present invention has discovered that a main reason for the unpleasant wearing experience in particular in obese persons is that the central body of the device does not assume a stable position in relation to the body of the wearer. In particular, the central body tends to pivot about the fastening points via which it is fastened to the belt, thereby causing discomfort and rendering it ineffective in sufficiently displacing the abdomen. Furthermore, the inventor has recognized that in order to achieve sufficient displacement of the abdomen, oftentimes the belt has to be tightened to a degree that causes discomfort. The inventor has discovered that these problems can be solved by an additional support structure that stabilizes the central body in its position relative to the body of the wearer and at the same acts on the central body so as to improve displacement of the abdomen by the central body. With the improved weight loss device, the wearer feels full faster, eats less and loses weight without diet pills or surgery

In addition, the inventor has discovered that mounting the central body about one finger above the navel of the wearer can improve wearing comfort and the appetite suppressing effect of the device.

According to one aspect of the present invention, an appetite suppression device which permits extended wear and is effective in causing weight loss, includes a central body having a portion that extends towards the body of the wearer when worn, a mounting structure for mounting the central body on a human being so as to exert pressure inwardly against a portion of the abdomen of the human being with the central body, the mounting structure being adapted to mount said central body between the navel and the sternum and approximately one finger above the navel of the human being; and a support structure arranged over at least a portion of the central body which faces away from the wearer.

According to another advantageous feature of the invention, the portion of the central body that extends toward the body of the wearer can be shaped convex in a direction of the body of the wearer.

According to another advantageous feature of the invention, the portion of the central body, which faces away from the wearer can be configured convex in a direction away from the wearer.

According to another advantageous feature of the invention, the support structure is adapted to exert a force on the central body, wherein at least a portion of the force is directed toward the body of the wearer.

According to another advantageous feature of the invention, the mounting structure and/or the support structure can be made of an elastic material and be configured in the form of a belt.

According to another advantageous feature of the invention, the support structure and the mounting structure can be constructed rigid.

According to another advantageous feature of the invention, the support structure and the mounting structure can each include a rigid portion and an elastic portion. For example, a portion of the support structure and/or the mounting structure can be constructed as a rigid plastic or metal brace and another portion of the support structure and/or the mounting structure can be made of an elastic material such as an elastic band or elastic belt.

According to another advantageous feature of the invention, the mounting structure can include at least two rigid portions, and a structure adapted for adjusting a position of the two rigid portions relative to each other can be arranged between the two rigid portions. This allows to for example to adjust the positions of the two rigid portions to each other so that a circumference embraced by the two rigid portions can be adjusted for example decreased to thereby move the central body further towards the body of the wearer, or increased to thereby allow the central body to move further away from the body of the wearer. An example for the rigid structures are curved plastic or metal part that roughly conform to the shape of the body of the wearer. An example for realizing the adjustment of the position of the two rigid portions relative to each other is to construct the two portions so that their ends are slidable over each other and to provide a clamp that forces the two rigid portions against one another in a region of the ends (as indicated by the arrows), thereby holding the two rigid portions in a desired position relative to each other. Other structures to effect the adjustable movement and to fix the two rigid portions in position relative to each other are conceivable.

According to another advantageous feature of the invention, the mounting belt and/or the support belt can contain cotton in the range from 1%-80%, and/or rayon in the range from 1%-80%.

According to another advantageous feature of the invention, the mounting belt and/or the support belt can contain cotton in the range from 10%-60%, and/or rayon in the range from 10%-60%.

According to another advantageous feature of the invention, the mounting belt and/or the support belt can contain cotton in the range from 20%-40%, and/or rayon in the range from 20%-40%.

According to another advantageous feature of the invention, the mounting belt and/or the support belt can contain 31% cotton and/or 31% rayon.

According to another advantageous feature of the invention, the support structure and or the mounting belt can have a material thickness in a range from 1 mm-10 mm.

According to another aspect of the invention, a method for suppressing appetite in human beings for the purpose of achieving effective weight loss including the steps of removably mounting the device according to the invention on a human being so that the central body is positioned between the sternum and the navel and approximately 1 finger above the navel of the wearer; and wearing the device for an extended period of time sufficient for effecting weight loss. Tests with patients conducted by the inventor have shown that positioning the central body one finger above the belly button of a patient (supra-umbilical region) is perceived particularly comfortable by the patients and leads to better weight loss results contrary to what was previously assumed in the prior art.

According to another advantageous feature of the invention, the device can be worn for at least several hours.

According to another advantageous feature of the invention, the device can be worn for a duration and with a frequency sufficient to achieve effective weight loss.

According to another advantageous feature of the invention, the protuberance can contain at least one hollow cavity, and the method can further include the step of tapping the body from time to time.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1 shows a cross sectional top view of an embodiment of the device according to the invention with a support structure as separate belt;

FIG. 2 shows a cross sectional top view of an embodiment of the device according to the invention with the support structure connected to the mounting belt;

FIG. 3 shows a top view of an embodiment of the device according to the invention with the central body being provided with a cover sleeve;

FIG. 4 shows a top view of an embodiment of the device according to the invention with the central body being provided with a cover sleeve;

FIG. 5 shows a top cross sectional view of another embodiment of the device according to the invention, wherein the support belt is formed as an extension of the mounting belt;

FIG. 6 shows a top cross sectional view of an embodiment of the device according to the invention with two rigid portions and an elastic portion arranged between the rigid portions;

FIG. 7 shows a top cross sectional view of an embodiment of the device according to the invention with two rigid portions and a device for fixing a position of the rigid portions relative to each other;

FIG. 8 illustrates steps of the method according to the invention;

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way. It should also be understood that the drawings are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

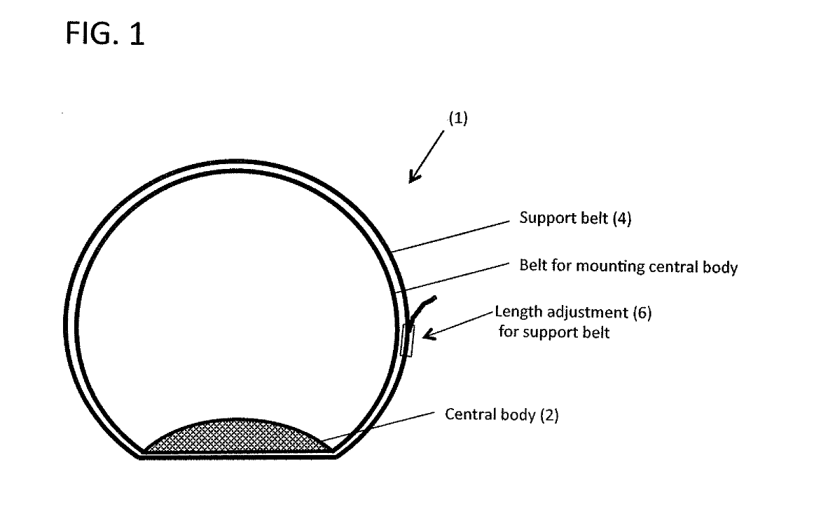

Turning now to the drawing and in particular to FIG. 1 there is shown an embodiment of a device 1 according to the invention including a central body 2, a mounting structure constructed as a belt 3 and connected to the central body 2 for mounting the central body 2 on a human being, and a support structure 4 in the form of a support belt 4. The support belt 4 can be used for stabilizing the position of the central body 2 in relation to the body of the wearer. In FIG. 1, the support belt 4 is separate from the belt 3 for mounting the central body 2 and is arranged over the central body 2 and the mounting belt 3 so as to cover the central body and the mounting belt along their circumferential extent around the waist of the wearer. The support belt 4 and the mounting belt 3 can have a length adjustment device 6 for adjusting a length of the support belt 4 or the mounting belt 3. The length adjustment device 6 can be any device that can be used to adjust the length of the support belt. For example the length adjustment device 6 can be provided in the form of a buckle.

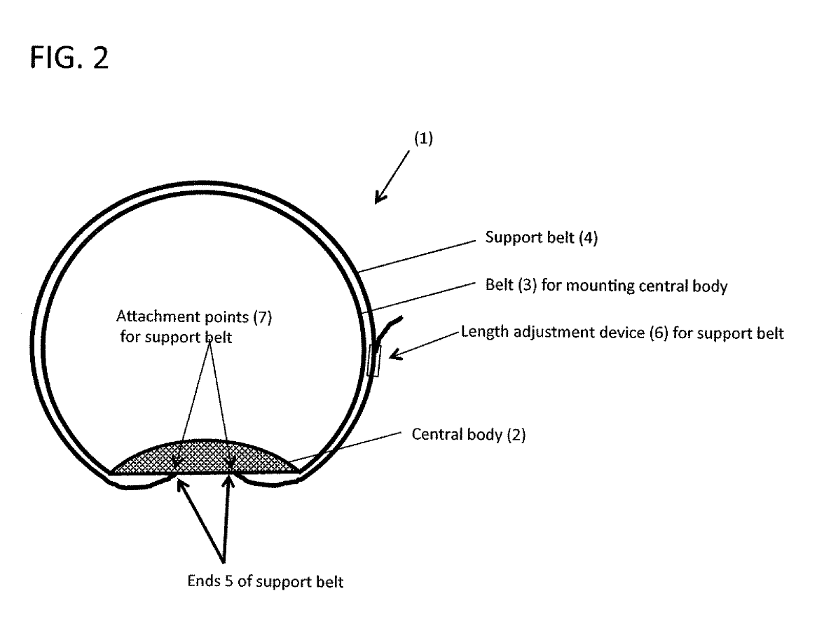

FIG. 2 shows another embodiment of the device according to the invention, wherein ends 5 of the support belt 4 are connected to the central body 2 at attachment points 7 on a front portion of the central body 2 which front portion faces away from the body of the wearer. The position at which the ends 5 of the support belt 4 are connected at the front portion of the central body 2 can vary along the length and width of the central body 2. Further, the means by which the ends of the support belt are connected to the portion of the central body can also vary and may include, but are not limited to, adhesives such as glue, hook and loop connectors, screws, or the like. Further, the ends 5 of the support belt 4 can be fastened to the portion of the central body 2 permanently or detachably.

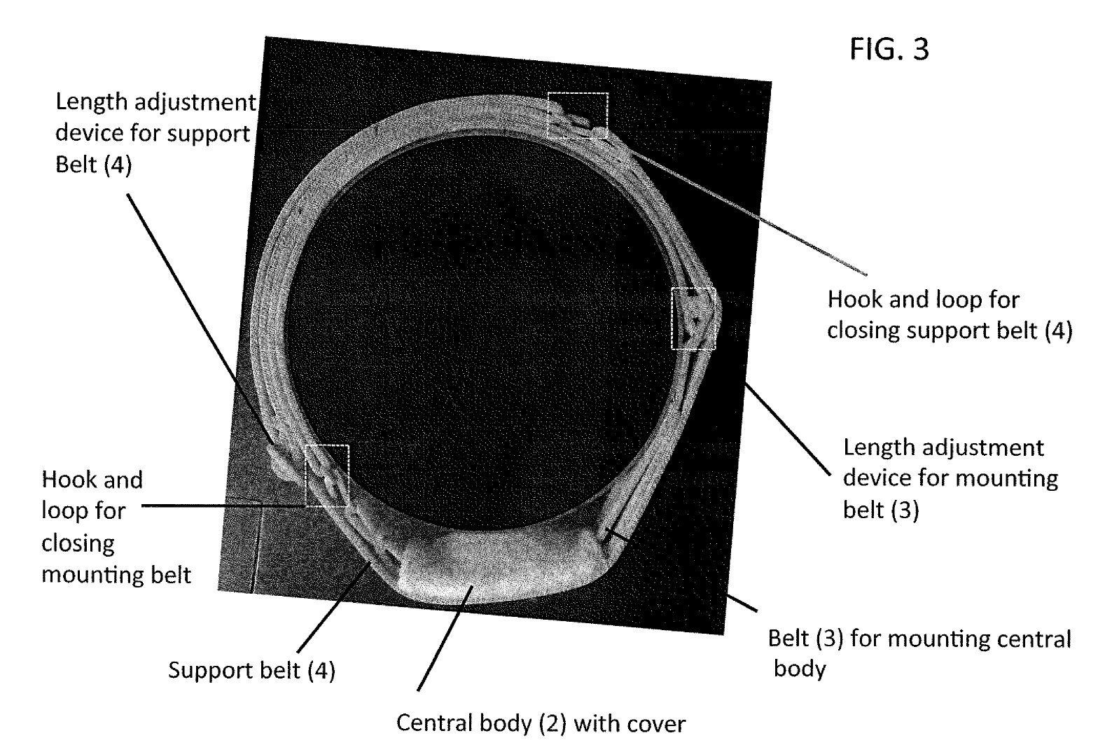

FIG. 3 shows a photographic representation of an embodiment of the device according to the invention illustrating the relative position of the central body 2, the mounting belt 3 and the support belt 4. The central body 2 is provided with a cover sleeve. The cover sleeve can be made of different materials. Advantageous are materials that do not irritate the skin of the wearer when directly exposed to the material. An example for a material for the cover sleeve is terry cloth. In the embodiment of FIG. 3 the support belt 4 and the mounting belt have a hook and loop structure for closing the support belt 4 or the mounting belt 3. FIG. 3 also shows the length adjustment device for the mounting belt 3 and the support belt 4.

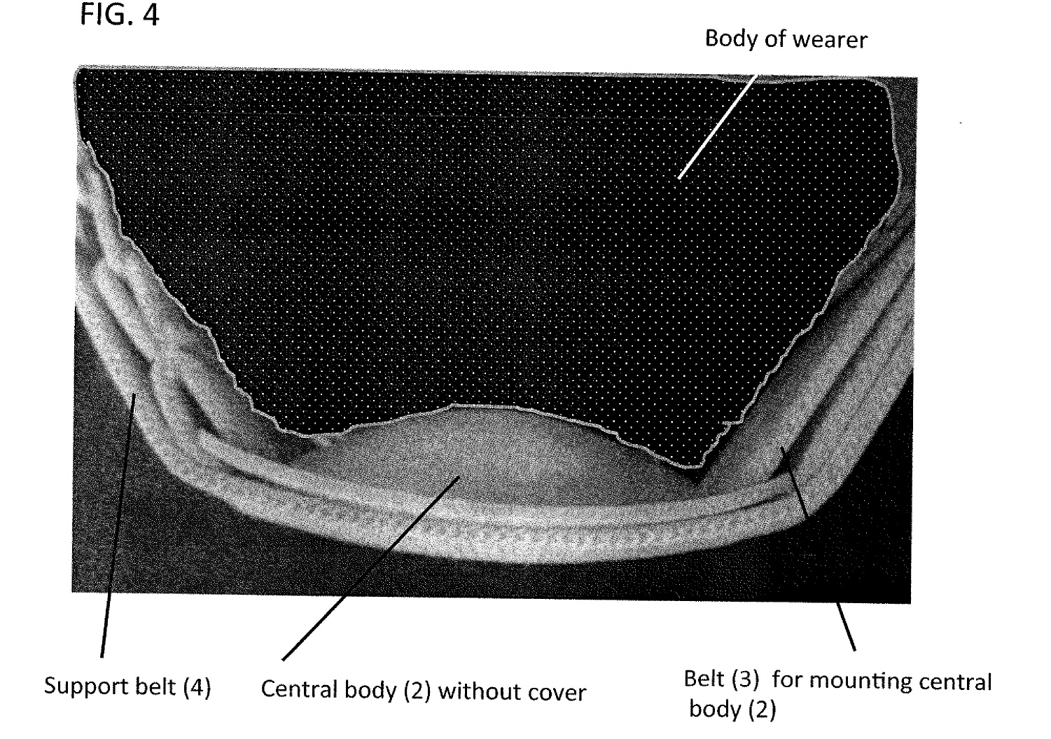

FIG. 4 shows the embodiment of the device according to the invention as shown in FIG. 3, without the cover sleeve covering the central body 2.

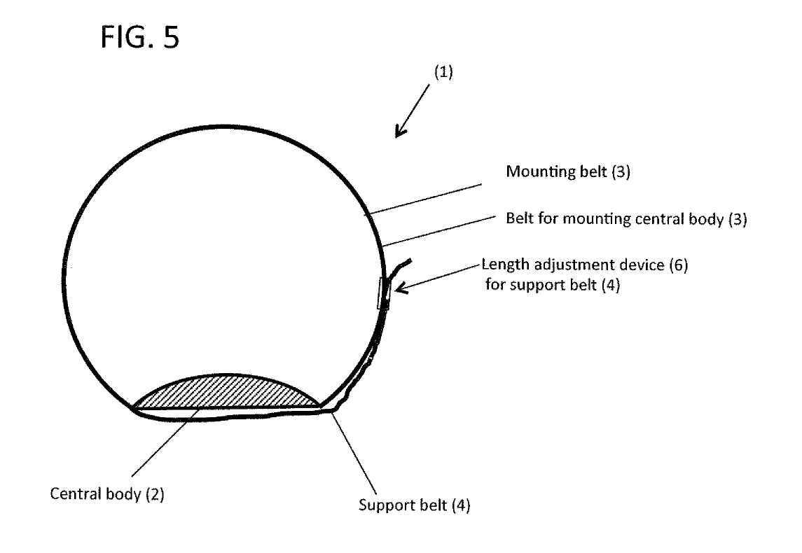

FIG. 5 shows another embodiment of the device 1 according to the invention in which the support belt 4 forms a part of the mounting belt. In the embodiment shown in FIG. 3, the support belt 4 is formed one-piece with the mounting belt 3 and forms an extension on one end of the mounting belt 3. The extension can be placed over the central body 2 and strapped around the waist of the wearer.

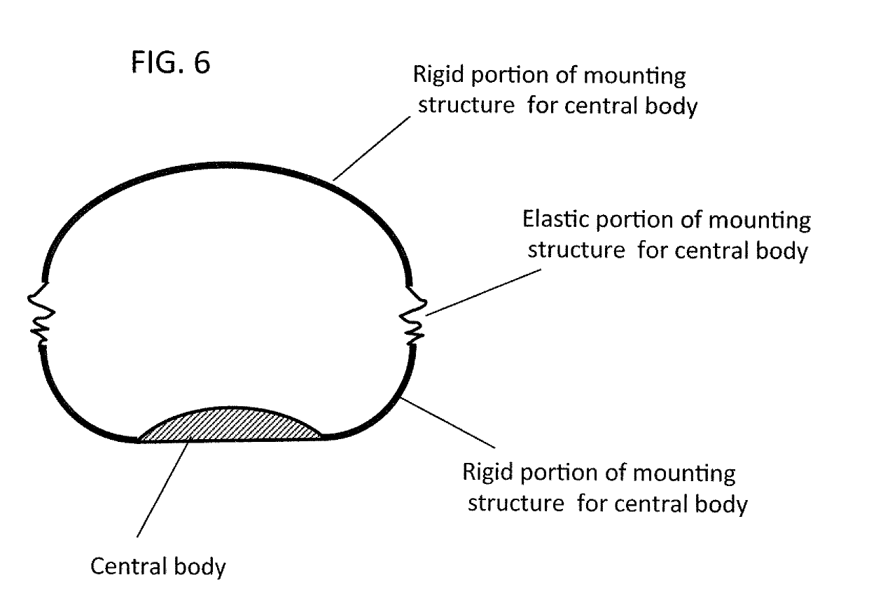

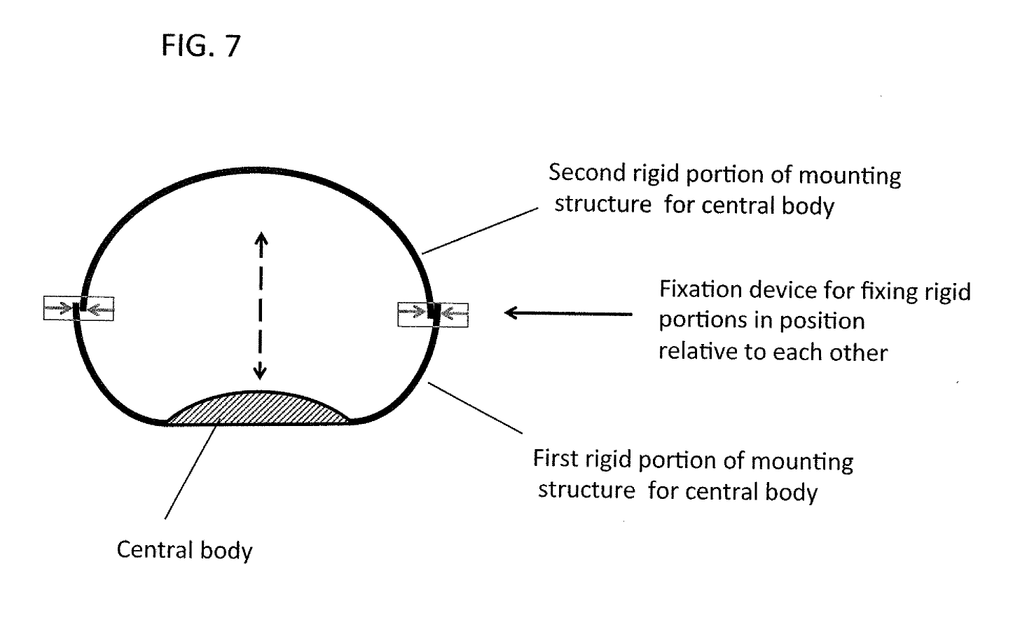

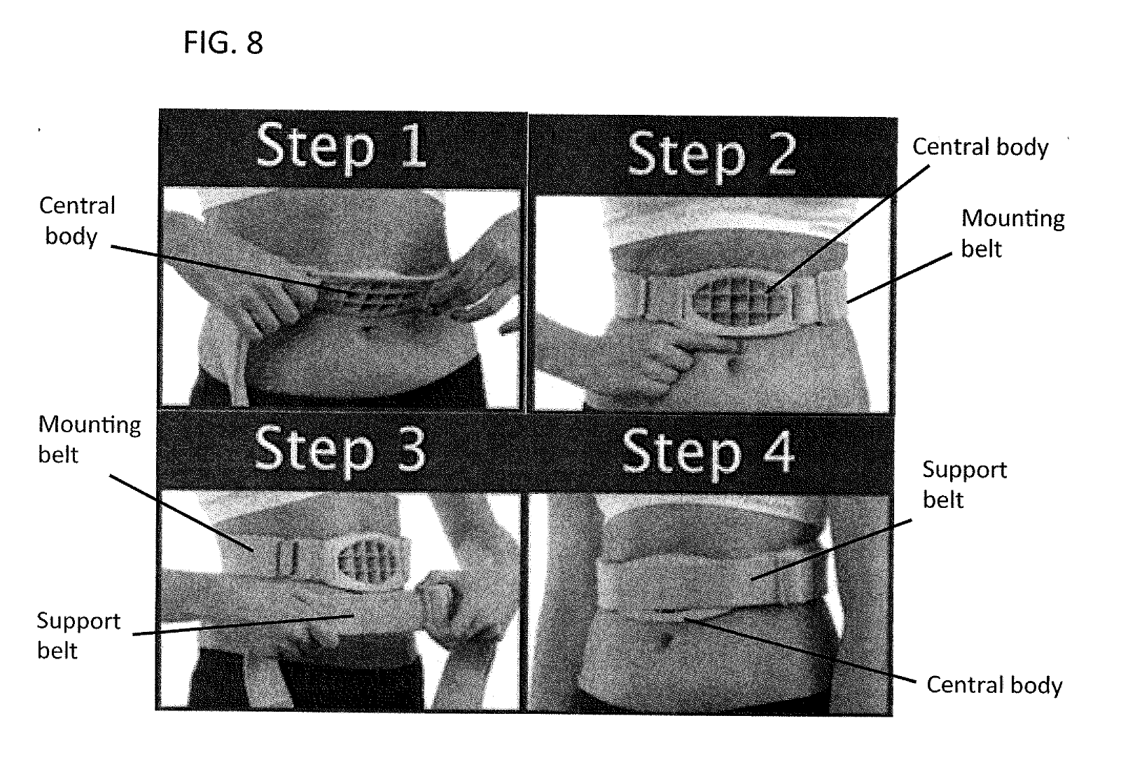

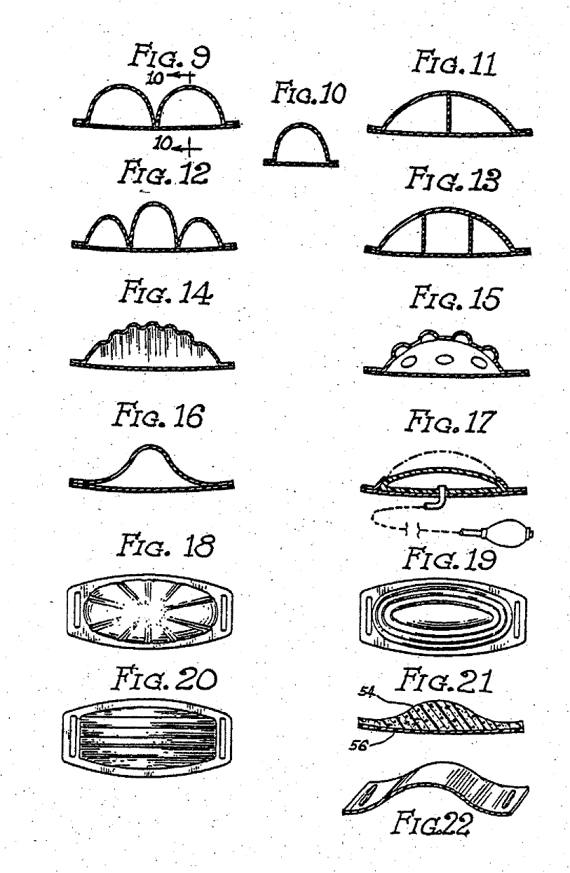

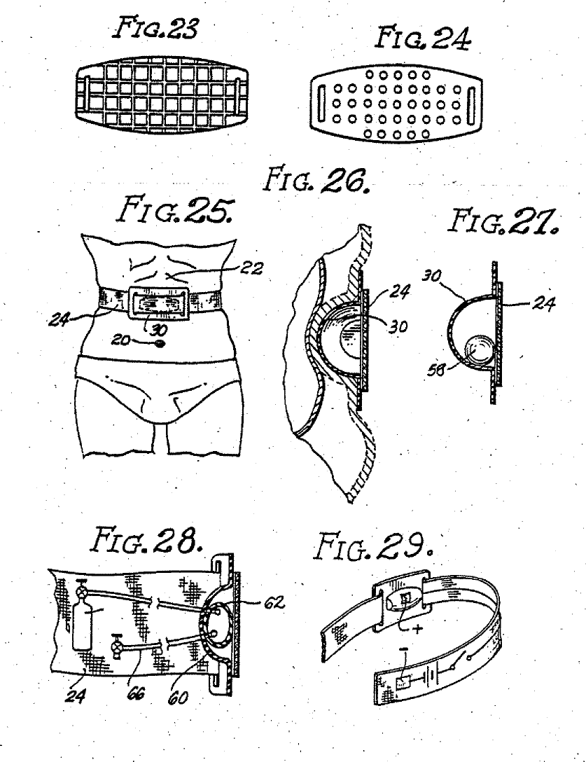

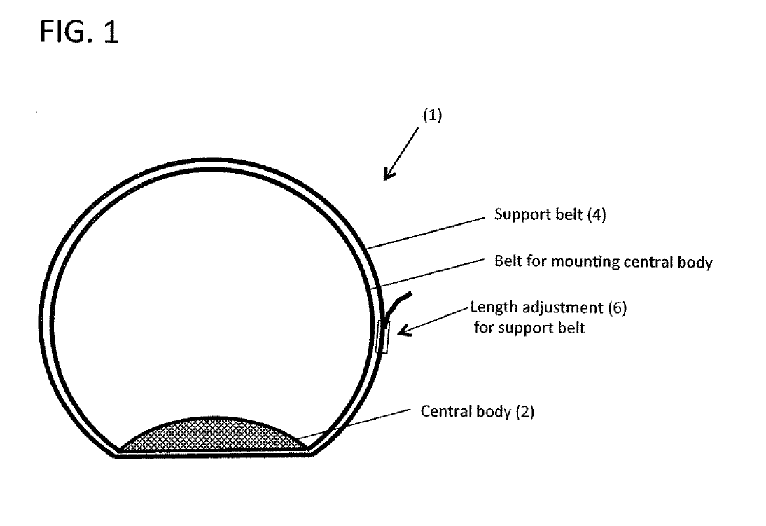

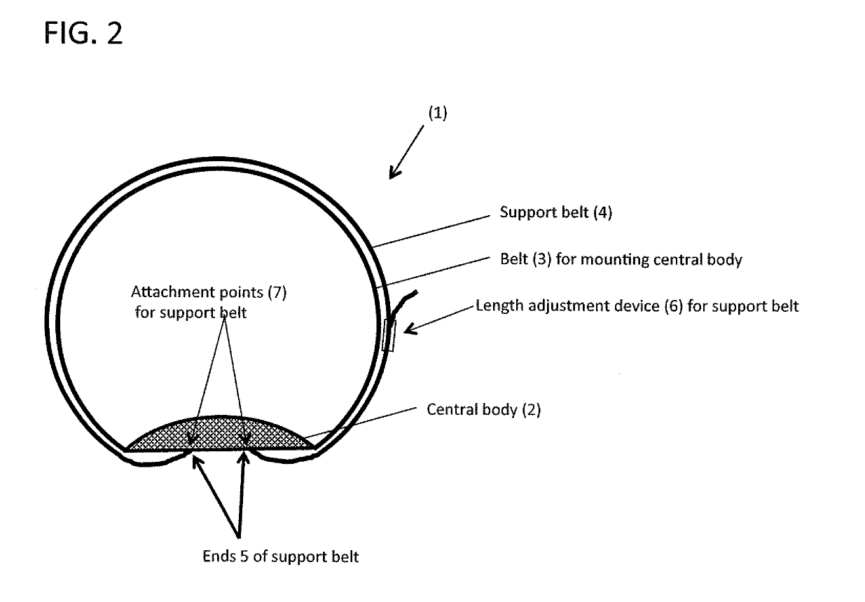

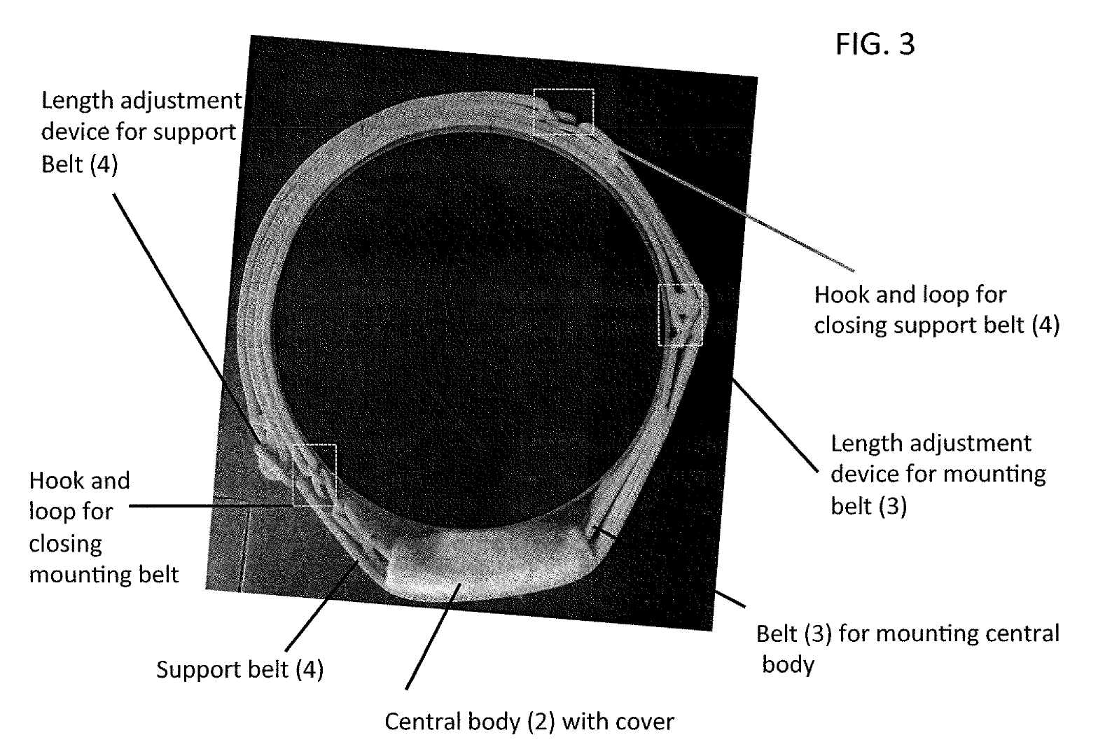

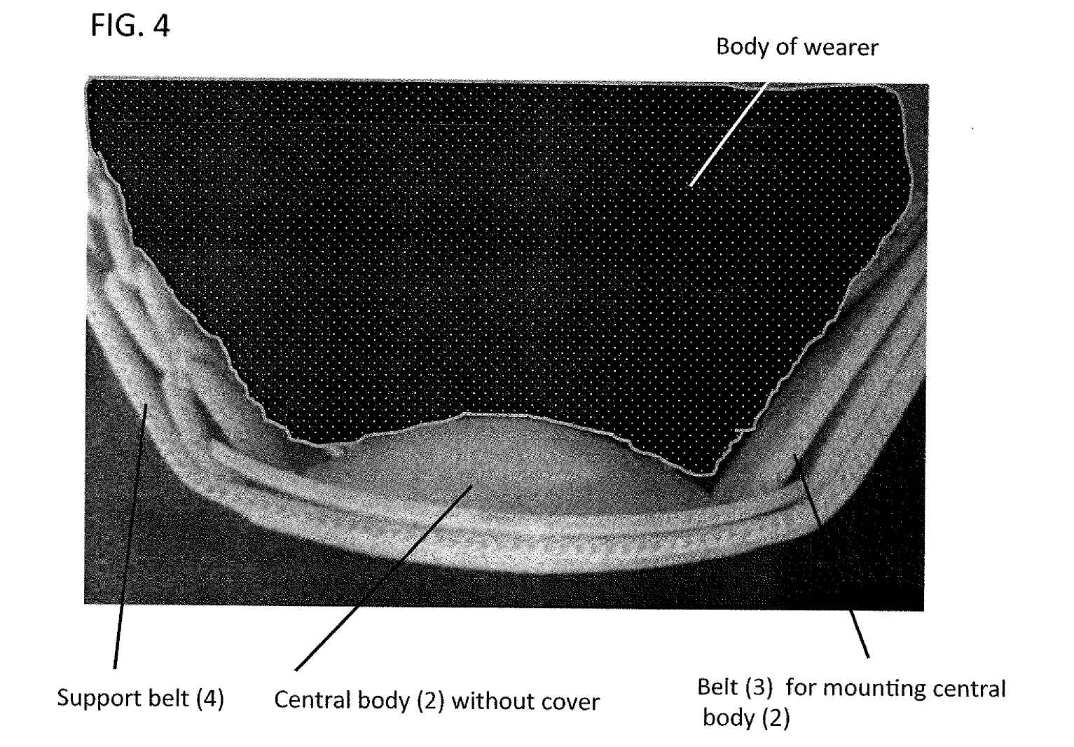

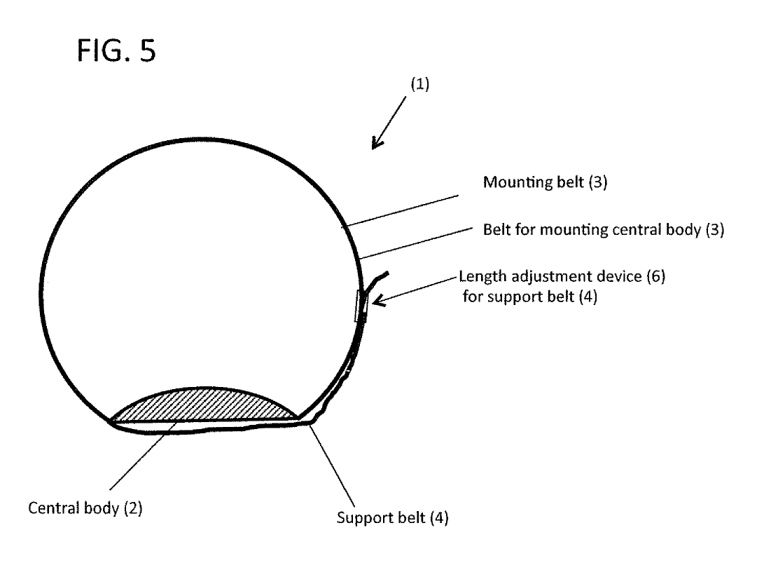

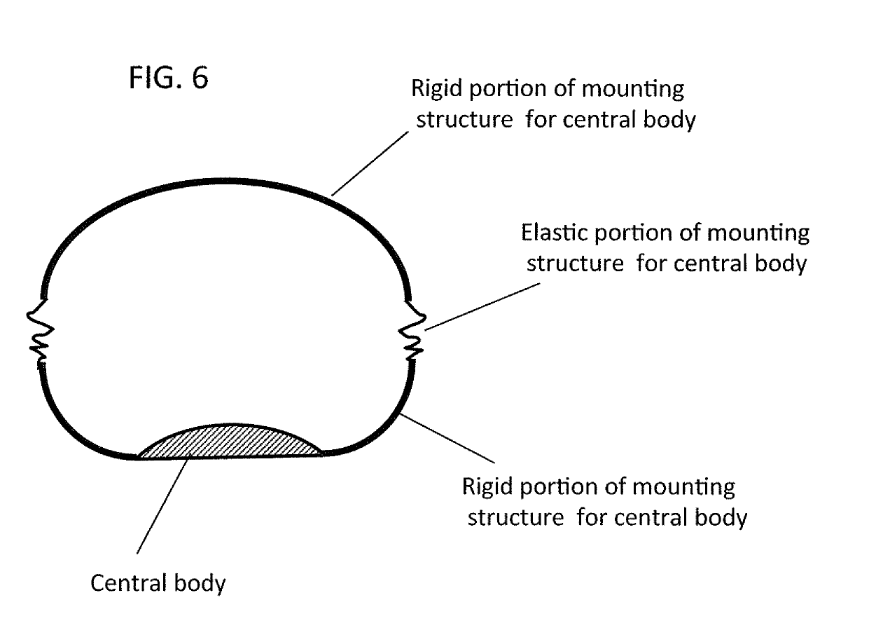

FIG. 6 shows an embodiment of the device according to the invention which includes two rigid portions that are connected by an elastic portion arranged in between them.

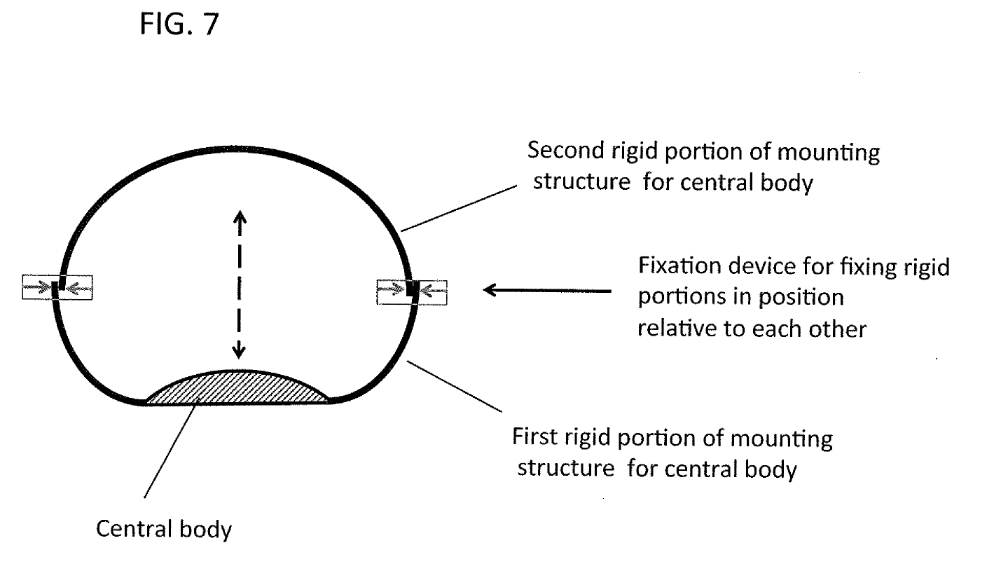

FIG. 7 shows another embodiment of the device according to the invention which includes a first and second rigid portions that can be moved relative to each other along the direction indicated by the dashed-lined double arrow away and toward the body of the wearer of the device. The rigid portions can be fixed at a desired position relative to each other with the fixation device which exerts a force on the ends of the rigid portions indicated by the opposing arrows.

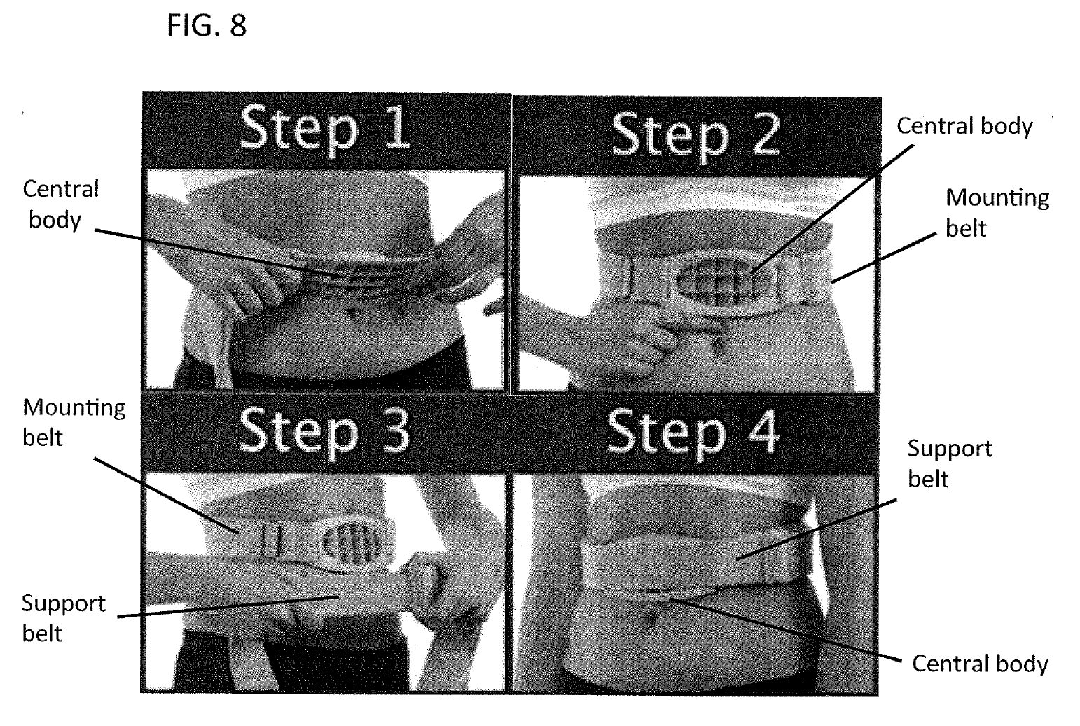

FIG. 8 illustrates possible steps to perform the method according to the invention using as an example an embodiment of the device according to the invention in which the mounting structure and the support structure are constructed as elastic belts. In step 1 the device is positioned so that the central body is located about one finger above the belly button of the wearer. In step 2 the device is fixed about the waist of the wearer. In steps 3 and 4 the support belt is applied over the central body and tightened about the waist of the wearer

A number of different embodiments have been illustrated in FIGS. 9-21, showing different approaches to the formation of the central body and similar variations.

FIGS. 9-13 illustrate different ways of providing the central body as several different resonant chambers rather than one. In FIGS. 9, 10, and 12, this is done by sculpting separate chambers, and in FIGS. 11 and 13, internal baffles define the different cavities. The resonance is helpful in transmitting an appetite suppressing vibration to the stomach. In another embodiment, any of the hollow versions of the body could be partially filled with water to stimulate the stomach with temperature difference and/or the vibrating sloshing action of the water.

FIGS. 14 and 15 illustrate central bodies which have been contoured by the provision of parallel ridges in FIG. 14 and spaced protrusions in FIG. 15. FIG. 16 is partially convex along its base area to provide a more accented point. FIG. 17 illustrates a means of inflating an inflatable version of the body, similar to the inflatable version illustrated in the parent application but using a valved squeeze bulb for pressure rather than a carbon dioxide cartridge. FIG. 18 illustrates how the FIG. 16 embodiment might look if it were star-shaped, and FIG. 19 illustrates a body contoured with concentric ovate ribs. FIG. 20 carries the same concept from FIG. 19 into parallel ribs, similar to FIG. 14. FIG. 21 illustrates a body composed of a solid mass of material 54 such as medium-density, semi-flexible XYLOFOAM foam, with a rigid backing 56. The backing is optional and could be omitted provided the foam were strong enough to support the belt. The embodiment shown in FIG. 22 is a single molded piece of plastic with slotted ends to engage the belt. This piece is a warped plane with no curve in the vertical dimension, like a ribbon or belt. FIG. 23 illustrates an embodiment having a striated, texturized surface. FIG. 24 illustrates a body that is perforated, to allow it to breathe in case the person had a perspiration problem. In any of the hollow embodiments, the hollow body could be all or partially filled with water or another liquid, for purposes of providing mass or heat conductivity, and in the case of a partially filled body, provide for a certain amount of sloshing around to stimulate the stomach.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed is:

1. An appetite suppression device, comprising:
a central body having a three-dimensional protuberance having an inwardly extending convex surface and an opposing outer surface which has a lesser degree of convexity;
a belt for mounting said protuberance on a human being so as to exert pressure inwardly against a portion of the abdomen of the human being with the protuberance, said belt being adapted to mount said protuberance between the navel and the sternum and approximately one finger above the navel of the human being; and
a support structure arranged over at least a portion of the central body and adapted for stabilizing a position of the central body in relation to the wearer and to exert a force acting inwardly on the central body.

2. The device of claim 1, wherein the support structure is connected to the belt.

3. The device of claim 2, wherein the support structure is formed one-piece with the belt.

4. The device of claim 1, further comprising a cover sleeve covering the central body.

5. The device of claim 4, wherein the cover sleeve is made of terry cloth.

6. The device of claim 1, wherein at least one of the mounting belt and the support structure includes at least one of cotton in the range from 1-80% and rayon in the range from 1-80%.

7. The device of claim 6 wherein at least one of the mounting belt and the support structure includes at least one of cotton in the range from 10%-60% and rayon in the range from 10-60%.

8. The device of claim 6, wherein at least one of the mounting belt and the support structure includes at least one of cotton in the range from 20%-40% and rayon in the range from 20-40%.

9. The device of claim 6, wherein at least one of the mounting belt and the support structure includes at least one of 31% cotton and 31% rayon.

10. The device of claim 1, wherein the support structure has a material thickness in a range from 1 mm-10 mm.

11. A method for suppressing appetite in human beings for the purpose of achieving effective weight loss comprising:
removably mounting on a human being a device comprising
a central body having a three-dimensional protuberance having an inwardly extending convex surface and an opposing outer surface which has a lesser degree of convexity;
a belt for mounting said protuberance on a human being so as to exert pressure inwardly against a portion of the abdomen of the human being with the protuberance, said belt being adapted to mount said protuberance between the navel and the sternum and approximately one finger above the navel of the human being; and
a support structure arranged over at least a portion of the central body and adapted for stabilizing a position of the central body in relation to the wearer and to exert a force acting inwardly on the central body; and
wearing the device for an extended period of time sufficient for effecting weight loss.

12. The method of claim 11, wherein the device is worn for at least several hours.

13. The method of claim 11, wherein the device is worn for a duration and with a frequency sufficient to achieve effective weight loss.

14. The method of claim 11, wherein the protuberance contains at least one hollow cavity, said method further comprising the step of tapping the central body from time to time.
